(19) United States
(12) Patent Application Publication
(10) Pub. No.: US 2024/0069036 A1
(43) Pub. Date: Feb. 29, 2024

(54) METHOD FOR IDENTIFYING NEOANTIGENS ON CANCER CELLS BY USING XENOANTIBODIES

(71) Applicant: Elliot Davis, Longwood, FL (US)

(72) Inventor: Elliot Davis, Longwood, FL (US)

(21) Appl. No.: 18/181,802

(22) Filed: Mar. 10, 2023

Related U.S. Application Data
(62) Division of application No. 16/243,161, filed on Jan. 9, 2019, now abandoned.
(60) Provisional application No. 62/617,240, filed on Jan. 14, 2018.

(51) Int. Cl. G01N 33/68; A61P 35/00; C07K 16/00; C07K 16/28; C07K 16/30; G01N 33/574

(52) U.S. Cl. CPC G01N 33/6854; A61P 35/00; C07K 16/00; C07K 16/2809; C07K 16/2812; C07K 16/2878; C07K 16/2896; C07K 16/30; G01N 33/57492; C07K 2317/14; C07K 2317/33; G01N 2500/10

(57) ABSTRACT

The presently disclosed subject matter relates to a method for finding and identifying sites or regions on cancer cells that are not possessed by normal cells (neoantigens). The method comprises creation of xenoantibodies which are able to specifically bind to cancer cell specific sites, and do not bind to, or cross react with normal cells. These neoantigens are used to plan and create cancer treatments.

This application is a division of U.S. patent application Ser. No. 16/243,161, filed Jan. 9, 2019, which claims the benefit of U.S. provisional application 62/617,240 filed Jan. 14, 2018, the contents of each of which are incorporated herein by reference in their entireties.

### TECHNICAL FIELD

The presently disclosed subject matter relates generally to a method for finding and identifying sites on cancer cells and methods of treatment thereof.

### BACKGROUND

This present disclosure provides a method for finding and identifying sites on cancer cells that are not possessed by normal cells (referred to here as neoantigens). The method comprises creation of xenoantibodies which are able to specifically bind to cancer cell specific sites. To create the xenoantibodies, cancer cells (or material derived from them) are taken from one species of animal and then introduced to the immune system of a different species of animal. The xenoantibodies then undergo a filtration process. By means of the filtration process, antibodies are selected that are specific to the cancer cells of a specific individual and do not bind to, or cross react with the normal cells of that individual. These cancer cell specific sites can be used as a target for various agents such as drugs or monoclonal antibodies to kill or inactivate cancer cells while not harming normal cells.

In previous attempts using antibody-based techniques, xenoantibodies were filtered using normal cells derived from individuals other than the ones from whom the cancer cells were derived (U.S. Pat. Nos. 4,798,719, 4,978,520 and 5,049,373). Since the cells of different individuals within a species differ in their immunogenic signature, this can result in incomplete filtration and therefore the failure to identify cancer-specific antigens. This presently described method uses cancer cells and normal cells derived from the same individual, significantly improving upon previous attempts and offering a superior method to find cancer neoantigens.

### SUMMARY

In accordance with the present invention, various embodiments of sample extraction devices and methods of use thereof are disclosed. In one embodiment, the present disclosure provides a method of identifying sites or regions (antigens) specific to cancer cells comprising the Steps of:
a. Cancer cells or their components are obtained from an individual (Animal One) of a given species of animal, wherein said individual may be of any species that is affected by cancer and the cells or their components may be obtained by any method known in the art.
b. The cancer cells or their components obtained from Animal One of Step a, are introduced to the immune system of an animal (Animal Two) of different species from animal One, wherein said cancer cells or their components may be introduced by any method known in the art.
c. Animal Two produces antibodies against the cancer cells or their components of Step b.
d. A blood sample comprising antibodies produced against the cancer cells or their components is obtained from Animal Two.
e. A solution (also referred to herein as immunizing solution) comprising antibodies present in the blood sample of Step d is prepared using any method traditionally used in the art. The solution is then exposed (i.e. via incubation) to normal cells, or their components, of Animal One, allowing antibodies-antigens binding. Thus, antibodies that bind said normal cells and/or components are removed from said solution.
f. The solution of Step e. comprising the remaining antibodies is introduced (i.e. via incubation) to a culture of the same cancer cells, or their components, of Step a, allowing antibodies-antigens binding. The antibodies that bind to the cancer cells or cancer cells components (to form antibody/antigen complexes) are selected for further analysis while the remaining solution is discarded.
g. The bound antibodies plus culture of cancer cells, or their components of Step f is fractionated to extract the antibody/antigen complexes. Fractionation is done using any suitable method known in the art.
h. The antibodies and the antigens in the extracted antibody/antigen complexes of Step g are separated using standard protocols.
i. The antigens of Step h are analyzed using standard methods such as, without limitation, methods for antigen identification, such as mass spectrometry.
Thus, sites or regions (antigens) specific to cancer cells are identified.

In some embodiments the present disclosure provides a method of using the antigens identified by the method described above to produce a medication, wherein the medication may be antibodies specific to said antigens or other therapeutic agents specific to those or related antigens.

In some embodiments the present disclosure provides a method of treating a subject in need comprising administering to said subject a therapeutically effective amount of a pharmaceutical composition comprising the medication which may be antibodies specific to said antigens or other therapeutic agents specific to those or related antigens.

In some embodiments the present disclosure provides a method of treatment of a subject in need comprising administering to said subject a therapeutically effective amount of a pharmaceutical composition comprising the medication which may be antibodies specific to said antigens or other therapeutic agents specific to those or related antigens, wherein the subject in need has cancer.

In some embodiments, the present disclosure provides A method of titration comprising the Steps of:
a. the solution of the solution claim 1 Step d. is serially diluted;
b. aliquot of each of the diluted solutions of Step a. is each added to a separate cell culture containing a fixed similar, or identical, number of cancer cells;
c. the antibodies in the said diluted solution are allowed to bind to the cancer cells, then supernatant of each of the cell cultures of Step b. is removed and each is added to a parallel, separate, cell culture containing a fixed similar, or identical, number of cancer cells;
d. fluorescent-conjugated secondary antibodies are added to the parallel culture of cancer cells of Step c. and biding is allowed;
e. the parallel culture of cancer cells of Step d. is inspected to assess the amount of the fluorescent secondary antibodies bound to the cancer cells; for comparison, the first sample of cancer cells is also incubated with fluorescent secondary antibodies and assessed for binding;
f. if, in Step e no signal from fluorescent secondary antibodies is detected in the first and second sample of cancer cells, then this would indicate that there were less antibodies than binding sites in the initial sample of cancer cells (Step b) and Steps a-e are be repeated with a more concentrated solution of antibodies with no change in the number of cancer cells. Steps a-e are repeated until a dilution is found wherein fluorescence can be detected in a certain dilution of the first plate, but not in the corresponding second culture; this would indicate that the amount of antibody attachment sites and antibodies are approximately equal;
g. when it is determined that the amount of antibody attachment sites and antibodies are approximately equal, an estimate of the volume of the diluted antibody containing solution that is required to saturate the antigenic sites on a single cancer cell is calculated by dividing the solution volume by the number of cells; and,
h. cancer cells and normal cells that are derived from the same individual and the same tissue type should contain virtually the same total number of antigens. Therefore, it can be assumed the volume of the diluted antibody containing solution that is required to saturate the antigenic sites on a single cancer cell is approximately the same as the volume necessary to saturate the amount of cross reacting antigenic sites on a single normal cell. Since the amount of antibody containing solution that will saturate the antigenic sites on a given number of cancer cells can now be calculated, the same ratio of antibodies to cells is used in the next step, which is incubation of the antibodies with the normal cells to remove cross-reacting antibodies, as described in claim 1, step e, and below. Therefore, with the above information, it can be easily ensured that the number of normal cells used for absorption, as described in claim 1 Step e and below, are adequate to absorb all the cross-reacting antibodies in the solution of claim 1 Step e and Step 4.

By “treating” is meant ameliorating at least one symptom of a condition or disease in a subject having the condition or disease (e.g., a subject diagnosed with cancer), as compared with an equivalent untreated control. Such reduction in the symptom (e.g., a reduction in tumor size or metastasis) is at least 5%, 10%, 20%, 40%, 50%, 60%, 80%, 90%, 95%, or 100%, as measured by any standard technique.

By “effective amount” or “therapeutically effective amount” is meant an amount of a molecule, compound or antibody required to treat, treat prophylactically, or reduce disease or disorder in a clinically relevant manner. For example, an effective amount of an active compound used according to the present invention varies depending upon the manner of administration, the age, body weight, and general health of the subject. Ultimately, the prescribers will decide the appropriate amount and dosage regimen.

By “subject”, or a “subject in need” is meant a human or non-human animal (e.g., a mammal).

While certain specific details of the construction and material selection have been disclosed herein, these will suggest many variations to those skilled in the art. It is not intended to limit this invention to the precise details disclosed herein. It will be apparent to those skilled in the art that many modifications and substitutions can be made to the preferred embodiments just described without departing from the spirit and scope of the invention as defined in the appended claims.

### BRIEF DESCRIPTION OF THE DRAWING

FIG. 1 illustrates the protocol of the titration method.

### DETAILED DESCRIPTION

In an embodiment of the present invention, cancer cells (or material derived from them) are taken from one species of animal and then introduced to the immune system of a different species of animal. The antibodies that are made in response to the introduced cancer cells (xenoantibodies) undergo a filtration process. By means of the filtration process, antibodies are selected that are specific to the cancer cells of a specific individual and do not bind to, or cross react with the normal cells of that individual. These antibodies are then introduced to a culture of cancer cells from that individual and the sites that they bind to are identified and analyzed. These cancer cell specific sites can be used as targets for various agents such as drugs or monoclonal antibodies in order to kill or inactivate cancer cells while not harming normal cells.

In the following description, references to human cancer are also applicable to cancer in any other species.

Virtually all cancer treatments that kill cancer cells also adversely affect normal cells and severely impact the health of people who suffer from cancer and are receiving those treatments. A primary goal of cancer research is to be able to tell the difference between cancer cells and normal cells, so that one can kill or neutralize the cancer cells without harming normal cells and thus, the patient.

Too often, the human immune system does not recognize its own cancer cells as being foreign or harmful and does not make antibodies against them. This is because the cancer cells are simply the body's own cells that have mutated in such a way as to reproduce in an uncontrolled fashion.

In contrast, it is generally accepted that a different species of animal will recognize human cancer cells as foreign or harmful and will make antibodies against them (xenoantibodies).

The present invention, instead of just relying on the human immune system to recognize human cancer cells, brings into play the spectacular variety and potential of the immune systems of the entire animal kingdom. Examples of the different types of animals are jawless fish, cartilaginous fish, bony fish, amphibians, reptiles, birds, and mammals.

Due to the fact that each non-human animal species' immune system is to varying degrees different from the human immune system, the potential for using other species' immune systems to find or detect cancer specific sites is enormous.

The essence of the present invention is to use xenoantibodies, which are created by non-human species of animals, against human cancer cells. These xenoantibodies are filtered against normal cells derived from the same individual from whom the cancer cells were derived, in order to define and characterize unique locations (regions) on the cancer cells of that individual. Once one finds one or more unique regions on the cancer cells that are not present on normal cells, those regions can be specifically targeted, to design treatments or medications which would kill or neutralize cancer cells, while not harming normal cells.

Unlimiting examples of such treatments or medications include antibodies, cytotoxic drugs and vaccines that target specifically the cancer cells while not affecting the normal cells. By “normal cell” is generally meant non-cancerous cells. In some embodiments, the normal cells and cancerous cells are derived from the same subject (i.e. human). In some further embodiments, the normal cells and cancerous cells are derived from the same subject (i.e. human) and are of the same body tissue type. Therefore, people will be treated for cancer without harming their normal cells and can regain their health.

In some embodiments, the present invention provides a method comprising of the following Steps:
Step 1: extract cancer cells from a person with cancer. Obtaining cancerous cells from a subject, for example a person, who has cancer can be done using any suitable method known in the art, such as without limitation, biopsy. In some embodiments, a solution which is formulized for injection into an animal (immunization) is then created, comprising the extracted cells or their components. In some embodiments, the said solution comprises at least one adjuvant. In some embodiments, the cells or components thereof are filtrated before addition of the adjuvant/s. In some embodiments, the immunizing solution is PBS (phosphate-buffered saline) based.
Step 2: inject the human cancer cells, or the immunizing solution, of in Step 1 into a non-human animal. Injections are administered according to protocols traditionally used in the art. The injections may be Subcutaneous (SC), Intramuscular (IM), Intraperitoneal (IP), or Intradermal (ID). Injections for routine antibody production may be administered subcutaneously in two to four sites per animal, generally on the back, away from the spine.

The animal will make antibodies against the cancer cells.

The animal will make different antibodies against many regions on the injected cancer cells. It is probable that many of the regions will be common to both the cancer cells and normal human cells, and some will be unique only to the cancer cells.

The animal's blood will also contain old antibodies that were made against foreign agents such as bacteria and viruses that the animal was previously exposed to.
Step 3: To harvest the antibodies produced, a blood sample is taken from the immunized animal of Step 2. In some embodiments, the antibodies are harvested by removing ascites fluid from the immunized animal of Step 2.

The sample (such as blood or ascites fluid) will contain antibodies made in response to the injected cancer cells and antibodies that were made prior to the injection of cancer cells. Therefore, it will contain various types of antibodies including:
1. antibodies that react (cross react) with both cancer and normal cells
2. antibodies that react ONLY with cancer cells
3. antibodies that are specific for the old agents (bacteria, viruses, etc.) that the animal was exposed to and were made prior to the exposure to cancer cells
Step 4: A solution comprising antibodies present in the sample of Step 3 is prepared using any protocol traditionally used in the art. Said solution, comprising all, or most, of the antibodies present in the blood sample of Step 3 is then incubated with a culture of normal human cells, and/or their components, that were derived from the same human from whom the cancer cells of Step 1 were extracted. Preferably, but not necessarily, the normal cells are derived from the same type of tissue that the cancer cells were extracted from. However, the normal cells may be extracted from any tissue of the body.
Step 5: antibodies that react with the normal cells will bind to them and therefore no longer be in solution. Those antibodies will then be filtered out of the solution and discarded. We are interested in finding antibodies that bind only to cancer cells.

As an example, a solution containing the antibodies mentioned in Step 3 is introduced to a culture of normal cells, and/or the cells' components (obtained from the animal of Step 1). The antibodies that have an affinity for normal cells or their components will bind to them and therefore be removed from the solution. The remaining solution may contain antibodies that have an affinity for the cancerous material obtained from the animal of Step 1, or antibodies that do not have an affinity for the aforementioned normal cells and were created by the non-human animal of Step 2 in response to old agents (described in article 3 of Step 3).
Step 6: put (i.e. incubate) the filtered (with the antibodies that bind to normal cells generally removed) solution of Step 5 in a culture of cancer cells or in a cancer cell lysate, derived from the same individual that the original cancer cells of Step 1 came from. The antibodies in the solution that are specific to cancer cells will bind to the cancer cells. Those antibodies are saved for further analysis.

The antibodies that were made to the old agents, as described in Step 3, will remain in solution. The solution can then be discarded.

Since the antibodies that would bind to normal cells or to agents that the animal was previously subjected to were discarded, the remaining antibodies that are saved should, entirely or for the most part, be specific for only cancer cells. Steps 7, 8, 9 as described below are performed to determine the identity of the cancer specific neoantigens.

The incubations of Step 4 and 6 are performed using any suitable protocol known in the art aimed to enable antibody/antigen binding. In some embodiments, the incubation is performed in room temperature, or 37° C. In some embodiments, the incubation length is in the range of 10 minutes to 72 hours. More specifically, the incubation is done “overnight”, within the range of 12-24 hours.
Step 7: fractionate the cancer cell culture or cancer cell lysate of Step 6 to extract the antibody/antigen complexes. Generally, the goal is to isolate those regions on the cancer cells where the xenoantibodies are bound to the cancer specific antigens.

As an example, the cancer specific antibodies are added to a cancer cell lysate (preferably the same cancer cells mentioned in Step 1 that have been broken up such that individual protein molecules are free in solution). Thus, the cancer specific antibodies are allowed to bind to their antigen targets. The antibody/antigen complexes can be separated from the rest of the solution comprising the cancer cell lysate (described above) using standard protocols.

The antibody/antigen complexes may be extracted from the cancer cell lysate using any suitable protocol known in the art, such as without limitation affinity chromatography, or co-immunoprecipitation. Co-IP generally involves capturing the immune complex, or precipitating it, for example, on a beaded support to which an antibody-binding protein is immobilized (such as Protein A or G), and any proteins not precipitated on the beads are washed away. Otherwise, the antibody/antigen are simply precipitated without the use of beads. Optionally then, the antigen (and antibody, if it is not covalently attached to the beads and/or when using denaturing buffers) may be eluted from the support and analyzed by sodium dodecyl sulfate-polyacrylamide gel electrophoresis (SDS-PAGE), often followed by western blot detection or mass spectrometryo verify the identity of the antigen.

In some embodiments, wherein the incubation of Step 6 is done with cancer cells, a cell-free lysate is prepared prior to extracting the antibody/antigen complexes. The conditions used for lysis should be gentle enough to retain the antibody binding sites but strong enough to quantitatively solubilize the antigen of interest. The lysis buffer may contain salt concentrations between 0-1M, nonionic detergent concentrations between 0.1-2%, divalent cation concentrations between 0-10 mM, EDTA concentrations between 0-5 mM, and pHs between 6-9. In addition, an antiprotease cocktail may be included.
Step 8: separate the xenoantibodies from the antigens using standard protocols, such as without limitation, western blot, or chromatography

At this point, the separated xenoantibodies of Step 8 may be further purified, e.g., using filtration, centrifugation and various chromatographic methods, such as HPLC or affinity chromatography, all of which purification techniques are well known to those of skill in the art. These purification techniques each involve fractionation to separate the desired antibody from other components of a mixture. Analytical methods particularly suited to the preparation of antibodies include, for example, protein A-Sepharose and/or protein G-Sepharose chromatography.
Step 9: analyze the neoantigens using existing methods for antigen identification, such as, without limitation, western blot, ELISA, gel shift assays, reporter assays, immunospectroscopy, or mass spectrometry.
Step 10: plan and create cancer treatments. There could be many different treatments possible including, but not limited to:
   The xenoantibodies, or monoclonal antibodies derived from them, could be used in a method of treatment to directly target and kill the cancer cells.
   Custom designed cytotoxic drugs could target the cancer specific antigens and kill the cancer cells that possess them.
   Directly inject the cancer specific antigens into the person with cancer and stimulate the body's own immune system to kill the cancer cells.
   Some combination of these therapeutic approaches, or others.

Antibodies or other therapeutic agents as exemplified in Step 10, specific to the antigens identified in Step 9, can be made in sufficient quantities to ultimately use for therapy.

In some embodiments of Steps 4 and 5, above, and Step e. of claim 1, the following titration method is used in order to optimize the antibody/normal cell ratio necessary to filter out cancer cell/normal cell cross-reacting antibodies.

As opposed to cancer cells, which are essentially immortal, normal cells tend to stop growing after a few divisions (Hayflick limit). It therefore may be difficult to obtain a sufficient number of normal cells to filter out antibodies that would cross react with both normal and cancer cells. In order to minimize the number of normal cells needed for cross-reacting antibody absorption, described Steps 4 and 5, above, and Step e. of claim 1, we can use cancer cells to determine the optimum amount of antibody containing solution needed to saturate the antibody binding sites on normal cells. This is because normal and cancer cells, that are extracted from the same animal and belong to the same tissue type, should be virtually identical in their antigenic properties. First, the dilution factor of the solution containing antibodies made against cancer cells (solution, also referred to as blood sample, of Step 3, above, and Step d. of claim 1) is ascertained, that is necessary to saturate the antigenic sites on a known number of cancer cells. Once the dilution factor is known, the volume of the diluted solution can be divided by the number of cancer cells that were used. Then one can calculate the volume necessary to saturate the antigenic sites on a single cancer cell. Since the cancer cells and their corresponding normal cells should be virtually equivalent in their antigenic signatures, the volume of antibody containing solution necessary to saturate a single normal cell would be virtually identical to that of a single cancer cell. Once the dilution factor is known, the optimum number of normal cells that would be needed to absorb all the antibodies that would tend to cross react, could be easily determined. Therefore, this would have the potential to drastically reduce the number of normal cells and also the number of absorption passes in the filtration process and thus remove the antibodies that cross react with normal cells. Preferably, in the following procedures, the cells should not be permeabilized, since the ultimate goal is to identify cancer-specific surface antigens.

In some embodiments, the general protocol of the above-mentioned titration method is described below. The goal is to determine the optimal quantity of antibodies, that were created against cancer cells, to introduce to a limited quantity of normal cells for the purpose of filtering out cross-reacting antibodies. To do this, we will use cancer cells as a proxy for the scarcer normal cells.

Protocol:
1. Prepare a solution of antibodies that were extracted from a rabbit, or any other animal, that was immunized against the cancer cells. It is recommended to begin this protocol with a highly diluted solution of antibodies, such as in the range of 1:5000 to 1:200,000, more specifically, the range may be 1:10,000 to 1:50,000, in order to conserve material.
2. Prepare several identical samples of the cancer cells at a fixed quantity/concentration.
3. Add a known volume of the antibody solution to the first sample of cancer cells. Incubate for 1 hour at room temperature or overnight at 4 degrees to allow binding.
4. Following the incubation, take a defined aliquot of the supernatant containing unbound antibodies and incubate it with a second sample of the cancer cells to allow binding
5. Add fluorescent secondary antibody to the second sample of cancer cells to visually detect if antibodies from the supernatant in step 4 have bound to the cells. Secondary antibody must also be incubated with a fresh sample of cancer cells in parallel, as a reference for non-specific secondary antibody binding.
6. If there is no fluorescent signal above background in the second sample of cancer cells, then the original solution of antibodies was completely bound to the first sample of cancer cells. Repeat steps #3-6 with higher starting concentrations of antibodies until there is a clear fluorescent signal above background in the second sample of cancer cells. This will reveal the ratio of antibodies to cells that just allows for saturation of binding sites in the first sample of cancer cells, and the appearance of unbound antibodies in the supernatant.
7. It can be assumed that the cancer cells and their corresponding normal cells have a virtually identical total number of antibody binding sites. We can then easily estimate the optimal volume and antibody concentration of the antibody sample that will be introduced, to the limited number of normal cells, for the purpose of filtering out cross-reacting antibodies.

The terms “antibody” as used herein, refer broadly to any immunological binding agent, including polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. The heavy-chain constant domains that correspond to the difference classes of immunoglobulins are termed α, δ, ε, γ and μ, respectively. The subunit structures and three-dimensional configurations of different classes of immunoglobulins are well known.

Generally, where antibodies rather than antigen binding regions are used in the invention, IgG and/or IgM are preferred because they are the most common antibodies in the physiological situation and because they are most easily made in a laboratory setting. The “light chains” of mammalian antibodies are assigned to one of two clearly distinct types: kappa (κ) and lambda (λ), based on the amino acid sequences of their constant domains. There is essentially no preference to the use of κ or λ light chains in the antibodies of the present invention.

For the purposes of the present disclosure, cell culture medium is a media suitable for growth of animal cells, such as mammalian cells, in in vitro cell culture. Cell culture media formulations are well known in the art. Typically, cell culture media are comprised of buffers, salts, carbohydrates, amino acids, vitamins and trace essential elements. The cell culture medium may or may not contain serum, peptone, and/or proteins. Various tissue culture media, including serum-free and defined culture media, are commercially available, for example, any one or a combination of the following cell culture media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series (JRH Biosciences, Lenexa, Kans.), among others. Cell culture media may be supplemented with additional or increased concentrations of components such as amino acids, salts, sugars, vitamins, hormones, growth factors, buffers, antibiotics, lipids, trace elements and the like, depending on the requirements of the cells to be cultured and/or the desired cell culture parameters.

Cell culture media may be serum-free, protein-free, and/or peptone-free. “Serum-free” applies to a cell culture medium that does not contain animal sera, such as fetal bovine serum. “Protein-free” applies to cell culture media free from exogenously added protein, such as transferrin, protein growth factors IGF-1, or insulin. Protein-free media may or may not contain peptones. “Peptone-free” applies to cell culture media which contains no exogenous protein hydrolysates such as animal and/or plant protein hydrolysates. Eliminating serum and/or hydrolysates from cell culture media has the advantage of reducing lot to lot variability and enhancing processing Steps, such as filtration. However, when serum and/or peptone are removed from the cell culture media, cell growth, viability and/or protein expression may be diminished or less than optimal. As such, serum-free and/or peptone-free cell culture medium may be highly enriched for amino acids, trace elements and the like. See, for example, U.S. Pat. Nos. 5,122,469 and 5,633,162.

Although there are many media formulations, there is a need to develop defined media formulations that perform as well or preferably better than those containing animal sera and/or peptones.

Defined cell culture media formulations are complex, containing amino acids, inorganic salts, carbohydrates, lipids, vitamins, buffers and trace essential elements. Identifying the components that are necessary and beneficial to maintain a cell culture with desired characteristics is an ongoing task. Defined basal media formulations which are supplemented or enriched to meet the needs of a particular host cell or to meet desired performance parameters is one approach to developing defined media. Identifying those components and optimum concentrations that lead to improved cell growth, viability and protein production is an ongoing task.

By cell culture or “culture” is meant the growth and propagation of cells outside of a multicellular organism or tissue. Suitable culture conditions for mammalian cells are known in the art. See e.g. Animal cell culture: A Practical Approach, D. Rickwood, ed., Oxford University Press, New York (1992). Mammalian cells may be cultured in suspension or while attached to a solid substrate. Fluidized bed bioreactors, hollow fiber bioreactors, roller bottles, shake flasks, or stirred tank bioreactors, with or without microcarriers, and operated in a batch, fed batch, continuous, semi-continuous, or perfusion mode are available for mammalian cell culture.

Mammalian cells, such as CHO cells, may be cultured in small scale cultures, such as for example, in 100 ml containers having about 30 ml of media, 250 ml containers having about 80 to about 90 ml of media, 250 ml containers having about 150 to about 200 ml of media.

Alternatively, the cultures can be large scale such as for example 1000 ml containers having about 300 to about 1000 ml of media, 3000 ml containers having about 500 ml to about 3000 ml of media, 8000 ml containers having about 2000 ml to about 8000 ml of media, and 15000 ml containers having about 4000 ml to about 15000 ml of media. Large scale cell cultures, such as for clinical manufacturing of protein therapeutics, are typically maintained for days, or even weeks, while the cells produce the desired protein(s).

During this time the culture can be supplemented with a concentrated feed medium containing components, such as nutrients and amino acids, which are consumed during the course of the production phase of the cell culture. Concentrated feed medium may be based on just about any cell culture media formulation. Such a concentrated feed medium can contain most of the components of the cell culture medium at, for example, about 5×, 6×, 7×, 8×, 9×, 10×, 12×, 14×, 16×, 20×, 30×, 50×, 100×, 200×, 400×, 600×, 800×, or even about 1000× of their normal amount. Concentrated feed media are often used in fed batch culture processes.

Temperature

The best temperature for culturing cells is related to the body temperature of the animal the cells were derived from. Typical temperatures for cell cultures derived from animals are: mammals 36° C. to 37° C., birds 38.5° C., cold blooded vertebrates such as fish, amphibians and reptiles between 15° C. and 26° C.

In some embodiments of any of the methods described herein, a polypeptide may be used in any of the methods of analyzing the antigens of the present disclosure. In some embodiments, the polypeptide is an antibody, such as T cell-dependent bispecific (TDB) antibody.

Molecular targets for antibodies include CD proteins and their ligands, such as, but not limited to: (i) CD3, CD4, CDS, CD19, CD11 a, CD20, CD22, CD34, CD40, CD79a (CD79a), and CD79|3 (CD79b); (ii) members of the ErbB receptor family such as the EGF receptor, HER2, HERS or HER4 receptor; (iii) cell adhesion molecules such as LFA-1, Macl, pl50,95, VLA-4, ICAM-1, VCAM and αv.β3 integrin, including either alpha or beta subunits thereof (e.g., anti-CD 11 a, anti-CD18 or anti-CD 1 ib antibodies): (iv) growth factors such as VEGF; IgE; blood group antigens; flk2/flt3 receptor; obesity (OB) receptor; mpl receptor; CTLA-4; protein C, BR3, c-met, tissue factor, β7 etc; (v) cell surface and transmembrane tumor-associated antigens (TAA), such as those described in U.S. Pat. No. 7,521,541, and (vi) other targets such as FcRH5, LyPD1, TenB2 and STEAP. In some embodiments, the antibody is an anti-CD20/anti-CD3 antibody. Other exemplar′ antibodies include those selected from, and without limitation, anti-estrogen receptor antibody, anti-progesterone receptor antibody, anti-p53 antibody, anti-HER-2/neu antibody, anti-EGFR antibody, anti-cathepsm D antibody, anti-Bcl-2 antibody, anti-E-cadherin antibody, anti-CA, 25 antibody, anti-CA15-3 antibody, anti-CA19-9 antibody, anti-c-erbB-2 antibody, anti-P-glycoprotein antibody, anti-CEA antibody, anti-retinoblastoma protein antibody, anti-ras oncoprotein antibody, anti-Lewis X antibody, anti-Ki-67 antibody, anti-PCNA antibody, anti-CD3 antibody, anti-CD4 antibody, anti-CD5 antibody, anti-CD7 antibody, anti-CD8 antibody, anti-CD9/p24 antibody, anti-CD 10 antibody, anti-CD ! 1 a antibody, anti-CD1 1c antibody, anti-CD 13 antibody, anti-CD14 antibody, anti-CD15 antibody, anti-CD 19 antibody, anti-CD20 antibody, anti-CD22 antibody, anti-CD23 antibody, anti-CD30 antibody, anti-CD31 antibody, anti-CD33 antibody, anti-CD34 antibody, anti-CD35 antibody, anti-CD38 antibody, a ti-CD4i antibody, anti-LCA/CD45 antibody, anti-CD45RO antibody, anti-CD45RA antibody, anti-CD39 antibody, anti-CD 100 antibody, anti˜CD95/Fas antibody, anti-CD99 antibody, anti-CD 106 antibody, anti-ubiquitin antibody, anti-CD71 antibody, anti-c-myc antibody, anti-cytokeratins antibody, anti-vimentin antibody, anti-HPV proteins antibody, anti-kappa light chains antibody, anti-lambda light chains antibody, anti-melanosomes antibody, anti-prostate specific antigen antibody, anti-S-100 antibody, anti-tau antigen antibody, anti-fibrin antibody, anti-keratins antibody, anti-TebB2 antibody, anti-STEAP antibody, and anti-Tn-antigen antibody.

By “cancer” is meant any condition diseases involving abnormal cell growth with the potential to invade or spread to other parts of the body. Cancer according to the present disclosure is, but not limited to Acute Lymphoblastic Leukemia (ALL), Acute Myeloid Leukemia (AML), Adrenocortical Carcinoma, Childhood Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Childhood Brain Cancer, Atypical Teratoid/Rhabdoid Tumor, Central Nervous System (Brain Cancer), Basal Cell Carcinoma of the Skin, Bile Duct Cancer, Bladder Cancer, Childhood Bladder Cancer, Bone Cancer (includes Ewing Sarcoma and Osteosarcoma and Malignant Fibrous Histiocytoma), Brain Tumors, Breast Cancer, Childhood Breast Cancer, Bronchial Tumors, Burkitt Lymphoma, Non-Hodgkin Lymphoma, Carcinoid Tumor (Gastrointestinal), Childhood Carcinoid Tumors, Carcinoma of Unknown Primary, Childhood Carcinoma of Unknown Primary, Cardiac (Heart) Tumors, Central Nervous System cancer, Atypical Teratoid/Rhabdoid Tumor, Embryonal Tumors, Germ Cell Tumor, Primary CNS Lymphoma, Cervical Cancer, Childhood Cervical Cancer, Cholangiocarcinoma, Bile Duct Cancer, Chordoma, Chronic Lymphocytic Leukemia (CLL), Chronic Myelogenous Leukemia (CIVIL), Chronic Myeloproliferative Neoplasms, Colorectal Cancer, Childhood Colorectal Cancer, Craniopharyngioma, Cutaneous T-Cell Lymphoma—(Mycosis Fungoides and Sézary Syndrome), Ductal Carcinoma In Situ (DCIS), Embryonal Tumors, Endometrial Cancer (Uterine Cancer), Ependymoma, Esophageal Cancer, Childhood Esophageal Cancer, Esthesioneuroblastoma, Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone-Malignant, Osteosarcoma, Gallbladder Cancer, Gastric (Stomach) Cancer, Childhood Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Childhood Central Nervous System Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Childhood Head and Neck Cancers, Heart Tumors, Hepatocellular (Liver) Cancer, Histiocytosis, Langerhans Cell, Hodgkin Lymphoma, Hypopharyngeal Cancer (Head and Neck Cancer), Intraocular Melanoma, Childhood Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Childhood Laryngeal Cancer and Papillomatosis, Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Childhood Melanoma, Melanoma, Intraocular (Eye), Childhood Intraocular Melanoma, Merkel Cell Carcinoma (Skin Cancer), Mesothelioma, Malignant Childhood Mesothelioma, Metastatic Cancer, Metastatic Squamous Neck Cancer with Occult Primary (Head and Neck Cancer), Midline Tract Carcinoma Involving NUT Gene, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Myelogenous Leukemia, Chronic (CIVIL), Myeloid Leukemia, Acute (AML), Chronic Myeloproliferative Neoplasms, Nasal Cavity and Paranasal Sinus Cancer (Head and Neck Cancer), Nasopharyngeal Cancer (Head and Neck Cancer), Childhood Nasopharyngeal Cancer, Neuroblastoma, Non-Hodgkin Lymphoma, NonSmall Cell Lung Cancer, Oral Cancer, Lip and Oral Cavity Cancer and Oropharyngeal Cancer (Head and Neck Cancer), Childhood Oral Cavity Cancer, Osteosarcoma and Malignant Fibrous Histiocytoma of Bone, Ovarian Cancer, Childhood Ovarian Cancer, Pancreatic Cancer, Childhood Pancreatic Cancer, Pancreatic Neuroendocrine Tumors (Islet Cell Tumors), Papillomatosis, Paraganglioma, Childhood Paraganglioma, Paranasal Sinus and Nasal Cavity Cancer (Head and Neck Cancer), Parathyroid Cancer, Penile Cancer, Pharyngeal Cancer (Head and Neck Cancer), Pheochromocytoma, Childhood Pheochromocytoma, Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Childhood Rhabdomyosarcoma (Soft Tissue Sarcoma), Salivary Gland Cancer (Head and Neck Cancer), Childhood Salivary Gland Tumors, Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Uterine Sarcoma, Sézary Syndrome (Lymphoma), Skin Cancer, Childhood Skin Cancer, Small Cell Lung Cancer, Small Intestine Cancer, Soft Tissue Sarcoma, Squamous Cell Carcinoma of the Skin, Squamous Neck Cancer with Occult Primary, Metastatic (Head and Neck Cancer), Stomach (Gastric) Cancer, Childhood Stomach (Gastric) Cancer, T-Cell Lymphoma, Cutaneous (Mycosis Fungoides and Sezary Syndrome), Testicular Cancer, Childhood Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Childhood Thyroid Tumors, Transitional Cell Cancer of the Renal Pelvis and Ureter (Kidney (Renal Cell) Cancer), Childhood Cancer of Unknown Primary, Ureter and Renal Pelvis, Transitional Cell Cancer, Kidney Renal Cell Cancer, Urethral Cancer, Endometrial Uterine Cancer, Uterine Sarcoma, Vaginal Cancer, Childhood Vaginal Cancer, Vascular Tumors (Soft Tissue Sarcoma), Vulvar Cancer, and Wilms Tumor and Other Childhood Kidney Tumors.

The therapeutic agents or compositions of the disclosure are given on a per diem basis but should not be interpreted as necessarily being administered on a once daily frequency. Indeed, the therapeutic agents, compositions, compound, salt or prodrug thereof, can be administered at any suitable frequency, for example as determined conventionally by a physician taking into account a number of factors, but typically about four times a day, three times a day, twice a day, once a day, every second day, twice a week, once a week, twice a month or once a month. In some situations, a single dose may be administered, but more typically administration is according to a regimen involving repeated dosage over a treatment period. In such a regimen the daily dose and/or frequency of administration can, if desired, be varied over the course of the treatment period, for example introducing the subject to the compound, composition, salt or prodrug thereof at a relatively low dose and then increasing the dose in one or more Steps until a full dose is reached. The treatment period is generally as long as is needed to achieve a desired outcome.

It will generally be found preferable to administer the (Active Pharmaceutical Ingredient-therapeutic agent, compounds and compositions of the present disclosure) API in a pharmaceutical composition that comprises the API and at least one pharmaceutically acceptable excipient. The excipient(s) collectively provide a vehicle or carrier for the API. Pharmaceutical compositions adapted for all possible routes of administration are well known in the art and can be prepared according to principles and procedures set forth in standard texts and handbooks such as those individually cited below:
USIP, ed. (2005) Remington: The Science and Practice of Pharmacy, 21st ed., Lippincott, Williams & Wilkins.
Allen et al. (2004) Ansel's Pharmaceutical Dosage Forms and Drug Delivery Systems, 8th ed., Lippincott, Williams & Wilkins.
Suitable excipients are described, for example, in Kibbe, ed. (2000) Handbook of Pharmaceutical Excipients, 3rd ed., American Pharmaceutical Association.

Examples of formulations that can be used as vehicles for delivery of the API in practice of the present disclosure include, without limitation, solutions, suspensions, powders, granules, tablets, capsules, pills, lozenges, chews, creams, ointments, gels, liposomal preparations, nanoparticulate preparations, injectable preparations, enemas, suppositories, inhalable powders, sprayable liquids, aerosols, patches, depots and implants.

For oral delivery, the API can be formulated in liquid or solid form, for example as a solid unit dosage form such as a tablet or capsule. Such a dosage form typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function.

Concerning all methods, the terms “a” and “an” are used to mean “at least one”, “at least a first”, “one or more” or “a plurality” of steps in the recited methods, except where specifically stated.

It is expressly contemplated that the methods described herein are not limited for the creation of antibodies specific to cancer antigens, but may be used for the creation and selection of target-specific antibodies against any desired antigen, such as without limitation, a viral antigen.

### Example 1

Detection of Antigens

For the detection of protein biomarkers various protein assays are available including, for example, antibody-based methods as well as mass spectroscopy and other similar means known in the art. In the case of antibody-based methods, for example, the sample may be contacted with an antibody specific for said antigen under conditions sufficient for an antibody-antigen complex to form, and then detecting said complex. Detection of the presence of the protein antigen may be accomplished in a number of ways, such as by Western blotting (with or without immunoprecipitation), 2-dimensional SDS-PAGE, immunoprecipitation, fluorescence activated cell sorting (FACS), flow cytometry, and ELISA procedures for assaying a wide variety of tissues and samples, including plasma or serum. A wide range of immunoassay techniques using such an assay format are available, see, e.g., U.S. Pat. Nos. 4,016,043, 4,424,279, and 4,018,653. These include both single-site and two-site or “sandwich” assays of the non-competitive types, as well as in the traditional competitive binding assays. These assays also include direct binding of a labeled antibody to a target antigen.

Sandwich assays are among the most useful and commonly used assays. A number of variations of the sandwich assay technique exist, and all are intended to be encompassed by the present invention. Briefly, in a typical forward assay, an unlabeled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride, or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g., 2-40 minutes or overnight if more convenient) and under suitable conditions (e.g., from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labeled with a reporter molecule. Depending on the amount of target and the strength of the reporter molecule signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by the reporter molecule. By “reporter molecule,” as used in the present specification, is meant a molecule which, by its chemical nature, provides an analytically identifiable signal which allows the detection of antigen-bound antibody. The most commonly used reporter molecules in this type of assay are either enzymes, fluorophores or radionuclide containing molecules (i.e., radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase, and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity.

When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art. However, other reporter molecules, such as radioisotope, chemiluminescent or bioluminescent molecules, may also be employed.

### Example 2

Production of serum and solutions containing xenoantibodies.

The following procedure or a variation can be used for any vertebrate.

A goat is inoculated by intramuscular injection with cancer cells or their derivatives using an intramuscular injection. Blood samples are drawn after an appropriate interval, such as two weeks, for initial assessment. In the optimized procedure, the goat is injected every week for four weeks, then at six weeks the animal is then bled to obtain the reagent.

Approximately 400 cc of blood is drawn from the goat under sterile technique. The area for needle extraction is shaved and prepared with betadine. An 18-gage needle is used to draw approximately 400 cc of blood from the animal. Of note is that the animal can tolerate approximately 400 cc of blood drawn without the animal suffering any untoward effects. The animal does not have to be sacrificed. The animal can then be re-bled in approximately 10 to 14 days after it replenishes its blood volume.

The presence of potentially useful antibodies is confirmed. Once the presence of such reagents is confirmed blood is then taken from the goat at between 4-6 weeks, and centrifuged to separate the serum. 300 ml of serum is then filtered to remove large clots and particulate matter. The serum is then treated with supersaturated ammonium sulfate (45% solution at room temperature) to precipitate antibodies and other material. The resulting solution is centrifuged at 5000 rpm for five minutes, after which the supernatant fluid is removed. The precipitated immunoglobulin is resuspended in phosphate-buffered saline (‘PBS buffer’, see Sambrook et. al. ‘Molecular cloning, A Laboratory Manual’, 1989) sufficient to re-dissolve the precipitate.

The solution is then dialyzed through a membrane with a molecular weight cut off of 10,000 Daltons. Dialysis is carried out in PBS buffer, changed every four hours over a period of 24 hours. Dialysis is carried out at 4° C.

After 24 hours of dialysis the contents of the dialysis bag are emptied into a sterile beaker. The solution is adjusted such that the mass per unit volume=10 mg per ml. The dilution is carried out using PBS. The resulting solution is then filtered through a 0.2 micron filter into a sterile container. After filtration, the solution is aliquoted into single doses of 1 ml and stored at −22° C. prior to use.

The reagent is then ready for use.

Changes may be made in this procedure, such as for example by varying the concentration of the ammonium sulfate or switching to other reagents. Similarly, the dialysis cut-off need not be at 10,000 Daltons.

### Example 3

In one embodiment of the titration method to determine optimal ratio of antibodies to cells the procedure described below is used.

To conserve material, the smallest size wells that the cancer cells will attach to and look healthy are used (i.e. 96-well or 24-well plates, if possible).

In some embodiments, a total of 12 wells, divided into 2 plates is needed (6 wells each).

The cancer cells are seeded into plate #1(6 wells total) at the standard density at which they are currently grown them, OR a recommended density for immunofluorescence detection.

The cancer cells are seeded into plate #2(6 wells total) exactly one day later (since this plate will be used one day later than the first).

The antibody mixture, basically the solution of Step 3, described above, or the solution of Step d. of claim 1, (also referred to herein as “blood sample”) is added to the first well of plate #1,such that the final dilution of the antibody mixture is 1 to 10. For example, if the total volume of media in the well is 1 mL, add 100 microliter of the antibody mixture+900 microliter of media (culture media).

Add the antibody mixture to successive wells of plate #1,such that the final dilution of the antibody mixture in the said wells, including the first well, is 1 to 100, 1 to 1000, 1 to 10,000 and lastly 1 to 100,000. The mentioned dilutions are intended as an example of the method, other suitable dilutions of the antibody mixture may be used, such as any preferred dilutions in the range of 1 to 1,000,000-1 to 1.

The sixth well of plate #1 should contain only culture media, without antibody mixture.

Incubate plate #1 overnight at 4 degrees to allow antibody binding.

Now, referring to plate #2. the culture media is removed from each well of plate #2, and directly replaced with the corresponding media of plate #1. For example, the culture media is discarded from plate #2, well #1, and directly replaced with the media from plate #1, well #1. The wells of plate #2 will now contain media with any unbound antibodies.

At this point, the wells of plate #1 are gently washed a few times with PBS, and incubated with a detectable, such as florescence-conjugate, secondary antibody. Either imaging is performed immediately, or fixative is added to preserve the binding of the said detectable antibodies for a later imaging and assessment.

Plate #2 is incubated overnight at 4 degrees to allow any antibodies still present in the media to bind to the cells.

Following the incubation of plate #2, the wells of plate #2 are gently washed a few times with PBS, and subsequently incubated with a detectable, such as florescence-conjugate, secondary antibody, and imaged in the same manner as plate #1 (either immediately, or following fixation) to assess the amount of secondary antibody binding.

The experiment can also be done in duplicate, for a total of 24 wells instead of 12.

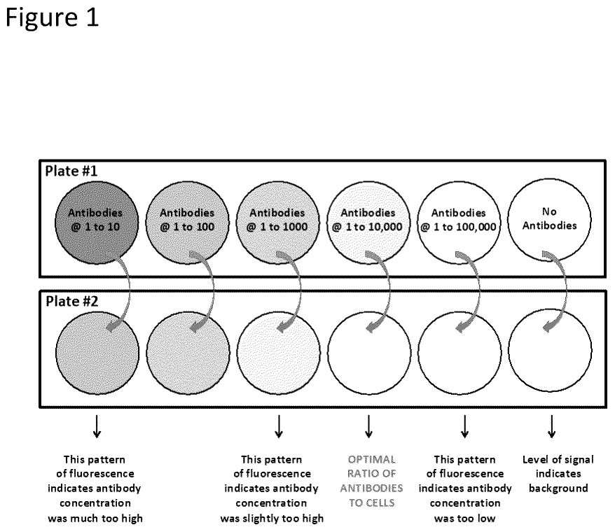

The protocol of Example 3 is schematically illustrated in FIG. 1.

1-5. (canceled)

6. A method of identifying a cancer-specific surface antigen comprising:
a. obtaining normal and cancer cells of the same tissue type from an individual animal having cancer (Animal One);
b. introducing the cancer cells to the immune system of an animal (Animal Two) sufficient for Animal Two to produce antibodies against antigens present on the surface of the cancer cells, wherein Animal One and Animal Two do not belong to the same species;
c. obtaining a blood sample from Animal Two comprising antibodies produced against antigens on the surface of the cancer cells;
d. exposing in vitro a solution comprising the antibodies to the normal cells obtained from Animal One such that the antibodies that bind to the normal cells are removed from the solution, thereby generating a filtered solution of antibodies;
e. introducing the filtered solution of antibodies to a culture or lysate of the same cancer cells of Step a sufficient to form antibody/antigen complexes;
f. extracting the antibody/antigen complexes from the culture or lysate of cancer cells; and
g. separating the antibodies and the antigens of the antibody/antigen complexes.

7. The method of claim 6, wherein Animal One is a human.

8. The method of claim 7, wherein Animal Two is a non-human animal species selected from a jawless fish, a cartilaginous fish, a bony fish, an amphibian, a reptile, a bird, and mammal.

9. The method of claim 8, wherein the mammal is a rabbit.

10. The method of claim 6, wherein obtaining the cancerous cells from Animal One comprises a biopsy.

11. The method of claim 6, wherein the introducing the cancer cells to the immune system of Animal Two comprising generating an immunization solution formulated for injection by Subcutaneous (SC), Intramuscular (IM), Intraperitoneal (IP), or Intradermal (ID) injection.

12. The method of claim 11, wherein the immunization solution comprises at least one adjuvant.

13. The method of claim 6, wherein the method further comprises labeling the antibodies of the filtered solution of antibodies prior to introducing the filtered solution of antibodies to a culture or lysate of the same cancer cells.

14. The method of claim 6, wherein extracting the antibody/antigen complexes from the culture or lysate of cancer cells comprises fractionating the antibody/antigen complexes from the components of the culture or lysate.

15. The method of claim 6, wherein extracting the antibody/antigen complexes from the culture or lysate of cancer cells comprises affinity chromatography or co-immunoprecipitation.

16. The method of claim 15, wherein the method further comprises detecting the presence of one or more of the antigens, wherein the detecting comprises a Western blot, an ELISA, a gel shift assay, a reporter assay, immunospectroscopy, or mass spectrometry.
